SEQUENCE LISTING (2 sequences)

SEQ ID NO: 1 - DNA, 609 bp, Listeria monocytogenes
atgacttacgaattacctaaattaccttatacttatgatgctttggagccgaattttgataaagaaaccatggaaattcactatacaaagcaccacaatacttatgtaacaaaactaaatgaagcggttgctggtcatcctgaacttgcaagcaaatctgcggaagaattagttactaacctagatagcgttcctgaagatattcgcggcgctgtccgtaaccacggtggcggtcatgctaaccatacattgttctggtctattcttagcccaaatggtggcggcgctccaactggcaatttaaaagcagcaatcgaaagcgaattcggtacttttgacgaatttaaagaaaaattcaatgcagcagctgcagcacgttttggttctggttgggcttggctagtagttaatgatggcaaattagaaatcgtttctacagctaaccaagattctccattaagcgatggcaaaacacccgttcttggcttagatgtttgggaacatgcttactaccttaaattccaaaaccgtcgtcctgaatatatcgaaacattttggaatgttattaactgggatgaagctaacaaacgctttgacgcagctaaataa

SEQ ID NO: 2 - protein, 202 aa, Listeria monocytogenes
MTYELPKLPYTYDALEPNFDKETMEIHYTKHHNTYVTKLNEAVAGHPELASKSAEELVTNLDSVPEDIRGAVRNHGGGHANHTLFWSILSPNGGGAPTGNLKAAIESEFGTFDEFKEKFNAAAAARFGSGWAWLVVNDGKLEIVSTANQDSPLSDGKTPVLGLDVWEHAYYLKFQNRRPEYIETFWNVINWDEANKRFDAAK

(19) United States
(12) Patent Application Publication
(10) Pub. No.: US 2006/0115494 A1
(43) Pub. Date: Jun. 1, 2006

(54) Recombinant BCG strains with attenuated immunosuppressive properties

(76) Inventors: Ronggai Sun, Ellicott City, MD (US); David Michael Hone, Rockville, MD (US); Jerald C. Sadoff, Washington, DC (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/284,890

(22) Filed: Nov. 23, 2005

Related U.S. Application Data
(60) Provisional application No. 60/631,986, filed on Dec. 1, 2004.

(51) Int. Cl. A61K 39/02; A61K 39/04; C12N 1/21

(52) U.S. Cl. 424/200.1; 424/248.1; 435/252.3

(57) ABSTRACT

Strains of Mycobacterium that have decreased immunosuppressive properties are provided. The Mycobacterium strains are genetically engineered to express but not secrete super-oxide dismutase (Sod). The presence of cytosol bound Sod allows replication and growth of the Mycobacterium, but does not result in attenuation of the host immune response. The Mycobacterium strains provide improved properties for use as vaccines.

### BACKGROUND OF THE INVENTION

1. Field of the Invention

The invention provides Mycobacterium strains that have reduced immunosuppressive properties. In particular, the invention provides Mycobacterium strains that are genetically engineered to express a super-oxide dismutase (Sod) that is not secreted by the Mycobacterium strains, and vaccine preparations containing the Mycobacterium strains.

2. Background

Mycobacterium tuberculosis (M. tb) has infected one-third of the world's population, causing active disease in 8 million and killing 1.6-2.2 million individuals every year, most of who live in the developing world. Tuberculosis (TB) is an epidemic of global proportions that is growing and becoming even more deadly as it intersects with the spread of HIV. TB is the number one killer of people with AIDS.

Bacille Calmette Guerin (BCG), the current widely used TB vaccine, was developed over 80 years ago and when tested has had widely variable rates of efficacy against pulmonary tuberculosis, including no efficacy in the last large field trial conducted in India (Fine et al., Vaccine, 16(20):1923-1928; 1998; Anonymous, Indian J Med Res., August; 110:56-69; 1999. Nonetheless, The World Health Organization (WHO) currently recommends BCG at birth or first contact with health services for all children (except those with symptoms of human immunodeficiency virus (HIV) disease/autoimmune disease syndrome (AIDS) in high TB prevalent countries. This policy is based on evidence that BCG protects against serious childhood forms of TB (Lanckriet et al., Int J Epidemiol, 24(5):1042-1049; 1995; Rodrigues et al., J Epidemiol Community Health 45(1): 78-80; 1991. Protection by BCG against TB beyond early childhood is a controversial subject with limited data giving mixed results. The high incidence of pediatric and adult TB in developing countries where infant BCG immunization is widely practiced, however, indicates that BCG as currently administered is not highly efficacious over the many years when people are at risk of TB disease. Thus, BCG is considered to be an inadequate public health tool for the intervention and control of TB.

Approximately 70 percent of humans exposed to TB organisms, and who have normal immune systems, do not become infected, and of those that do become infected only about 5 percent develop disease within the first two years. The majority of infected individuals suppress the infection, which is associated with the development of robust cellular immune responses to M. tb antigens. An additional 5 percent later reactivate when immunity declines. Both primary and reactivation disease are much more common in people with HIV/AIDS, again emphasizing the role of immunity in preventing and controlling infection.

### SUMMARY OF THE INVENTION

Because most humans are able to control TB, there is good reason to hope that by inducing long lasting immunity of the appropriate kind it should be possible to develop effective vaccines that prevent initial infection after exposure, prevent early progression to disease, prevent reactivation from the latent state and prevent relapse after treatment. Ultimately, it is the combination of systematic vaccine use plus chemotherapeutic intervention that will eventually eliminate M. tb as a human pathogen.

In light of the critical role childhood BCG vaccination is thought to play in preventing acute TB, it is difficult to replace BCG in trials to evaluate candidate TB vaccines without overwhelming evidence that the new TB vaccine is a superior product. The problem is that M. tb is a human-specific pathogen and animal models only mimic parts of the host-pathogen interaction. Thus, definitive evidence that a new TB vaccine possesses improved potency can only be obtained from controlled field trials in humans. This reality has led many investigators to conclude that a key step toward an improved TB vaccine will be to enhance the immunogenicity of BCG.

One example of such a strategy is to eliminate or attenuate the immunosuppressive properties of BCG. There is a broad consensus that the Mycobacteria, including BCG, manipulate the host response, thus preventing elimination (Flynn et al. Annu Rev Immunol. 19:93-129; 2001; Mariotti et al., Infect Immun. 72(8): 4385-92; 2004). Many factors have been implicated in the regulation of host responses (Flynn et al., supra, 2001); yet not all of the factors impart such properties when tested in vivo using mutants that lack the factor. More recently, however, evidence has emerged that the iron-cofactored super-oxide dismutase (SodA), of Mycobacterium, encoded by the sodA gene (Rv3846) appears to inhibit innate host immune responses that play a critical role in the initiation of adaptive immune responses (Edwards et al., Am J Respir Crit Care Med. 190(1): 115-22; 2001). However, it has been difficult to establish evidence that SodA inhibits innate host responses, since this enzyme is essential for the growth of Mycobacterium organisms, including M. tb and BCG (Edwards et al. supra, 2001). To overcome this hurdle, a M. tb strain was isolated that harbored a plasmid that expressed antisense soda RNA and decreased the total level of SodA. This strain proved to b e attenuated in mice and more immunogenic than the parental M. tb strain (Edwards et al. supra, 2001). Thus, although this approach has merit and may be applicable to BCG, the current method for the manipulation of SodA levels is less ideal as it requires antibiotic-resistant shuttle vectors, an unacceptable shortfall. Moreover, it would be difficult to undergo large-scale production of a live BCG strain harboring an antisense RNA, as such constructs would be susceptible to mutation and loss of activity. Ideally, rBCG strains with modified SodA expression should be generated so that the modification is irreversible. Heretofore, this hurdle has proven insurmountable, which is probably due to the fact that SodA is essential for the growth of M. tb and BCG, and the tools that are known to those skilled in the prior art to manipulate expression of soda (e.g. point mutations that alter the regulation/activity or antisense RNA that decrease expression of SodA) are prone to reversion.

The prior art has thus far failed to provide a BCG strain with reduced ability to manipulate the host response. In particular, the prior art has thus far failed to provide a BCG strain with a stable means to modify SodA expression, and that does not interfere with growth of the recombinant BCG (rBCG).

One aspect of this invention is the provision of rBCG strains with a reduced capability to manipulate the response of host cells. These novel rBCG strains thus do not inhibit innate host immune responses, thereby permitting a more robust immune response to the presence of the bacteria. Such strains of rBCG are especially advantageous for use in vaccine preparations, where a robust immune response to the bacteria that are administered is highly desirable. In particular, these novel rBCG strains produce a Sod enzyme that permits growth of the bacterium, but that is not secreted and so does not inhibit innate host immune responses. Thus, the rBCG strains can be grown in culture in order to prepare vaccines, and can grow and reproduce in a host organism that is vaccinated with a preparation containing the rBCG strains. In such a vaccinated host organism, a robust immune response is mounted to the rBCG because the non-secreted Sod enzyme does not enter the host cell cytoplasm and therefore does not inhibit the host cell immune response.

The invention provides a Mycobacterium that is genetically engineered to contain and express a functional super-oxide dismutase (Sod) enzyme that is not secreted by the Mycobacterium. The functional Sod enzyme is isolated from a bacterial species such as, for example, Salmonella enterditis, Escherichia coli, Shigella flexneri, Listeria monocytogenes, and Corynebacterium spp. In one embodiment, the functional Sod enzyme is SodA from Listeria monocytogenes EGD-e. The Mycobacterium may be an attenuated Mycobacterium such as BCG, and the Mycobacterium may further contain and express a transgene.

The invention also provides a method of decreasing the immunosuppressive properties of a Mycobacterium, comprising the step of genetically engineering the Mycobacterium to contain and express a cytosol-bound Sod enzyme. The functional Sod enzyme is isolated from a bacterial species such as, for example, Salmonella enteriditis, Escherichia coli, Shigella flexneri, Listeria monocytogenes and Corynebacterium spp. In one embodiment, the functional Sod enzyme is SodA from Listeria monocytogenes EGD-e. The Mycobacterium may be an attenuated Mycobacterium such as BCG, and the Mycobacterium may be further genetically engineered to contain and express a functional transgene.

The invention also provides a vaccine preparation, comprising a Mycobacterium that is genetically engineered to contain and express a functional Sod enzyme that is not secreted by the Mycobacterium. The functional Sod enzyme is isolated from a bacterial species such as, for example, Salmonella enteriditis, Escherichia coli, Shigella flexneri, Listeria monocytogenes and Corynebacterium spp. In one embodiment, the functional Sod enzyme is SodA from Listeria monocytogenes EGD-e. The Mycobacterium may be an attenuated Mycobacterium such as BCG, and the Mycobacterium may be further genetically engineered to contain and express a functional transgene. The vaccine preparation of claim 16, wherein said Mycobacterium is further genetically engineered to contain and express a functional transgene.

The Mycobacterium may be further genetically engineered to: escape the endosomal compartment and enter the cytoplasm; induce apoptosis; and/or express cytokines.

The invention further provides a method of treating cancer in a patient in need thereof. The method comprises the step of administering to said patient a vaccine preparation, comprising a Mycobacterium that is genetically engineered to remove its native super-oxide dismutase (Sod) enzyme and to contain and express a functional super-oxide dismutase (Sod) enzyme from a heterologous bacterial genus, wherein said functional Sod enzyme from said heterologous bacterial genus is not secreted by said Mycobacterium. The functional Sod enzyme from said heterologous bacterial genus may be SodA from Listeria monocytogenes EGD-e, and the Mycobacterium may be an attenuated Mycobacterium such as BCG. In some embodiments, the Mycobacterium is further genetically engineered to contain and express a functional transgene. The Mycobacterium may be further genetically engineered to: escape the endosomal compartment and enter the cytoplasm; induce apoptosis; and/or express cytokines.

### BRIEF DESCRIPTION OF THE DRAWINGS

FIG. 1. The map for suicide vector pAF120. The denotation for each of the DNA segments as follow: L-flank and R-flank: left and right flanks of sodA gene respectively; P_{Ag85B} is the promoter sequence of antigen 85B gene (i.e. Rv1886c); SodA is the gene encoding Listeria monocytogenes EGD-e Superoxide dismutase (accession number: NT01LM1550); Resbs is the resolvase binding sequence (gene bank accession number: X03526). Hyg is the gene encoding hygromycin B phosphotransferase (bank accession number: DQ005458) which confers hygromycin resistance. Pₕₛₚ₆₀ is the promoter sequence of the heat shock protein gene (i.e. Rv0440); SacB is the gene (gene bank accession number: Y489048) encoding levansucrase, which confers the bacteria sensitivity to sucrose; OriE is the pUC origin of replication (gene bank accession number: AY234331); aph is aminoglycoside phosphotransferase gene (gene bank accession number: X06402), which confers Kanamycin resistance for the plasmid; MCS is the multiple cloning sites for the indicated restriction enzymes. Note that the cassette between two PacI sites can be replaced with other endosomalytic enzyme genes when applicable.

FIG. 2. Flow chart for the principle steps of allele exchange as described in the text.

FIG. 3. The map for helper vector pAF121. The denotation for each of the DNA segments as follow: Pₕₛₚ₆₀ is the promoter sequence of heat shock protein gene (i.e. Rv0440); SacB is the gene (gene bank accession number: Y489048) encoding levansucrase, which confers the bacteria sensitivity to sucrose; OriE is the pUC origin of replication (gene bank accession number: AY234331); aph is aminoglycoside phosphotransferase gene (gene bank accession number: X06402), which confers Kanamycin resistance for the plasmid; oriM is the origin of replication in mycobacterium (gene bank accession number: M23557). P_{Ag85B} is the promoter sequence of antigen 85B gene (i.e. Rv1886c); tnpR is the transposon gamma-delta resolvase gene (gene bank accession number: J01844.

FIG. 4. Sequence of SodA from Listeria monocytogenes EGD-e A, nucleic acid sequence; B, amino acid sequence.

### DETAILED DESCRIPTION OF THE PREFERRED EMBODIMENTS OF THE INVENTION

As discussed above, evidence has emerged that the iron-cofactored SodA, encoded by the soda gene of Mycobacterium appears to inhibit innate host immune responses, which play a critical role in the initiation of adaptive immune responses. Reduction or elimination of SodA, therefore, would enhance the immunogenicity of BCG and other mycobacteria through increased immune recognition potentially through crosspriming mechanisms. However, soda cannot be simply eliminated, as this secreted protein is essential to the survival of this organism. Indeed, previous attempts to reduce or eliminate SodA production by BCG or M. tuberculosis have failed because elimination of SodA is a lethal mutation (Dussurget et al. Infect Immun., 69: 529-533; 2001; Edwards et al. supra, 2001).

Thus far, the only approach to tackle this problem that has met some success is the use of antisense soda RNA, which decreases the total level of SodA (Edwards et al. supra, 2001). However, this approach is not ideal as it requires antibiotic-resistant shuttle vectors that bear the unacceptable shortfalls outlined in the preceding section, and it would be difficult for an rBCG strain harboring an antisense RNA to undergo large-scale production, because such constructs would be susceptible to mutation and loss of activity. Ideally, rBCG strains with modified SodA expression should be generated so that the modification is irreversible. Heretofore this hurdle has proven insurmountable.

The present invention provides the novel approach of constructing rBCG strains that reduce levels of Sod in the host cell, without introducing lethal mutations, and without involving the use of antisense RNA. The strategy used herein entails the use of a form of Sod that is not secreted by the Mycobacterium strain. Such a form, therefore, does not reduce the level of oxdative burst metabolites, an innate host response, in the endosome in which Mycobacterium resides, but would be able to perform the normal housekeeping functions of Sod, which are vital to the survival of Mycobacterium.

Allelic exchange is used to construct Mycobacterium strains, such as rBCG and attenuated M. tuberculosis strains that express functional non-secreted forms of Sod, wherein the wild-type soda gene of the Mycobacterium strain is replaced by sequences that express a functional cytoplasmic-bound Sod. Procedures for allelic exchange in Mycobacterium are described in the Examples section below.

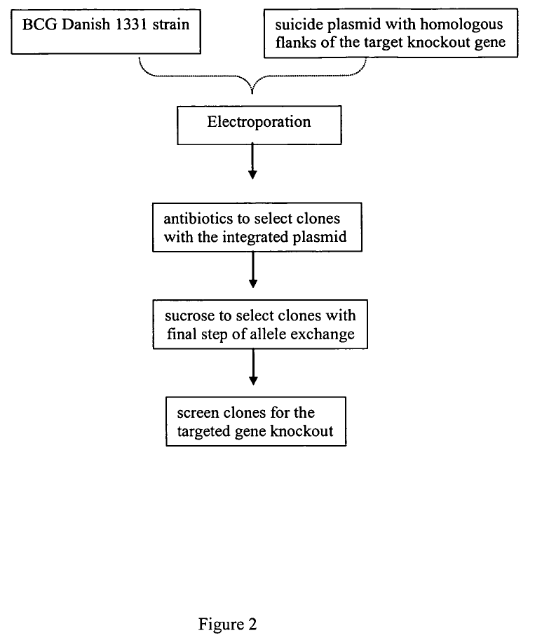

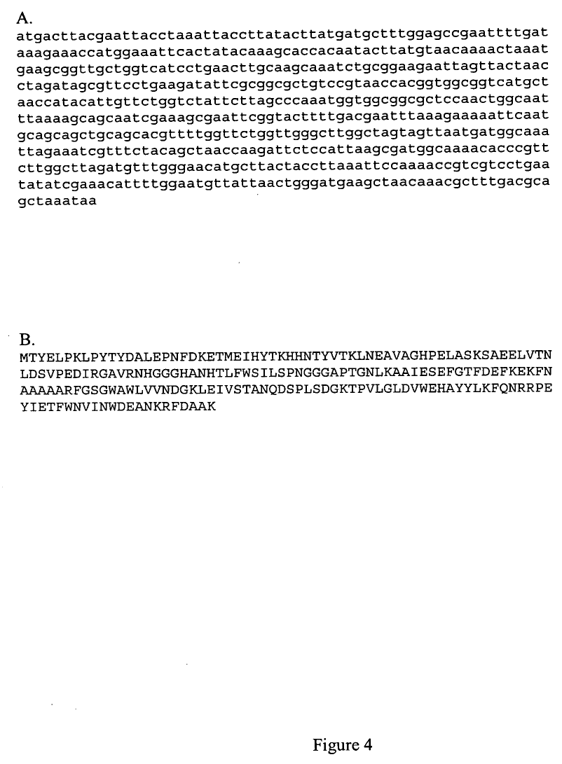

The particular sequence that encodes the non-secreted functional Sod employed in the present invention is not critical thereto if it conforms to the criterion that it is not secreted when expressed in Mycobacterium. Examples include, but are not restricted to, secA1 secreted SodA lacking a leader peptide from the Salmonella enteriditis (Genbank accession no. 1068147), Escherichia coli (Genbank Accession No. 1250070) or Shigella flexneri (Genbank accession no. 1079977) or alternatively a SodA protein that is naturally non-secreted such as the SodA from Listeria monocytogenes EGD-e (Genbank Accession No. 986791, and FIGS. 2A and B; sequences in FIG. 4). Such recombinant Mycobacterium strains do not produce extracellular Sod and thus do not suppress host immune responses, yet they do express intracellular Sod, thereby enabling survival of the rBCG organisms.

In a preferred embodiment of the invention, the recombinant Mycobacteria are attenuated, as exemplified by BCG. However, those of skill in the art will recognize that other attenuated and non-attenuated Mycobacteria exist which would also be suitable for use in the present invention. Examples of additional types of Mycobacteria include but are not limited to M. tuberculosis strain CDC1551 (See, e.g. Griffith et al., Am. J. Respir. Crit. Care Med. August; 152(2):808; 1995), M. tuberculosis strain Beijing (Soolingen et al., 1995), M. tuberculosis strain H37Ra (ATCC#: 25177), M. tuberculosis strain H37Rv (ATCC#: 25618), M. bovis (ATCC#: 19211 and 27291), M. fortuitum (ATCC#: 15073), M. smegmatis (ATCC#: 12051 and 12549), M. intracellulare (ATCC#: 35772 and 13209), M. kansasii (ATCC#: 21982 and 35775) M. avium (ATCC#: 19421 and 25291), M. gallinarum (ATCC#: 19711), M. vaccae (ATCC#: 15483 and 23024), M. leprae (ATCC#:), M. marinarum (ATCC#: 11566 and 11567), M. microtti (ATCC#: 11152). etc.

Examples of attenuated Mycobacterium strains include but are not restricted to M. tuberculosis pantothenate auxotroph strain (Sambandamurthy, Nat. Med. 2002 8(10):1171; 2002), M. tuberculosis rpo V mutant strain (Collins et al., Proc Natl Acad Sci USA. 92(17):8036; 1995), M. tuberculosis leucine auxotroph strain (Hondalus et al., Infect. Immun. 68(5):2888; 2000), BCG Danish strain (ATCC # 35733), BCG Japanese strain (ATCC # 35737), BCG, Chicago strain (ATCC # 27289), BCG Copenhagen strain (ATCC #: 27290), BCG Pasteur strain (ATCC #: 35734), BCG Glaxo strain (ATCC #: 35741), BCG Connaught strain (ATCC # 35745), BCG Montreal (ATCC # 35746).

By “functional Sod” or “functional form” of an enzyme we mean a form of the enzyme that is produced by the rBCG bacterium that exhibits the characteristic catalytic activity for which the native or natural (“wild type”) enzyme is known in its native organism. The activity of the functional form of the enzyme produced by the rBCG is, in general, at least about 50 percent of the usual activity of the wild type enzyme when assayed under standard conditions as recognized by those of skill in the art for the enzyme. Activity of the enzyme may be determined by measuring any physical observable, such as catalysis of a substrate, or production of an effect of the enzyme, such as escape of rBCG from endosomes. Preferably, the activity is at least about 50%, 60%, 70%, 80%, 90%, 100%, or more of the standard activity of the enzyme.

By “functional variant” of an enzyme, we mean a polypeptide whose amino acid sequence is at least about 70% homologous to that of a wild type “reference” enzyme, and which retains the functional activity (as described above) of the wild type enzyme. The amino acid sequence of the reference wild type enzyme is typically used as a starting point for mutations and alterations that are carried out by genetic engineering.

Preferably, a functional variant is a polypeptide with an amino acid sequence that is about 75%, 80%, 85%, 90%, 95% or more homologous to the reference amino acid sequence of the enzyme of interest of which it is a functional variant. Such functional variants retain the characteristic activity of the enzyme. Such functional variants include but are not limited to polypeptide sequences in which one or more conservative amino acid substitutions have been made. Conservative amino acid substitutions are well known to those of skill in the art, and include, for example, the substitution of one positively charged amino acid for another, one negatively charged amino acid for another, one hydrophobic amino acid for another, etc. Variant polypeptides may contain one or more of such substitutions, provided the resulting variant polypeptide retains enzymatic activity as defined herein. Functional variants also encompass other changes to the primary sequence of the polypeptide of interest. Examples of changes include but are not limited to deletions and additions of amino acids, or the modification of amino acids (e.g. chemical modifications such as sulfonation, deamidation, phosphorylation, hydroxylation, etc.). Such changes may be the result of genetic engineering of a reference amino acid sequence of an enzyme of interest, or may be the result of post-translational modifications of the enzyme, or both. Further, functional variants of an enzyme may be the result of natural mutations such as those that occur between analogous enzymes with the same or similar activities that are isolated from different strains of a species, or from different species, or from different individual organisms within a species. Such naturally occurring variants may also serve as the enzyme of interest, and the amino acid sequence of such a natural variant may serve as the reference sequence. In any case, all such functional variants of a reference enzyme retain the activity of the enzyme, as described herein.

Sequence homologies as described herein are not intended to include heterologous amino acid sequences that are derived from sources other than the reference sequence and which are attached to or included in the polypeptide sequence of an enzyme for various other purposes. Examples of such heterologous sequences include but are not limited to sequences that facilitate polypeptide isolation (e.g. histidine tags), sequences that facilitate secretion or localization of the polypeptide within the cell, (e.g., various leader sequences), and sequences that code for glycosylation sites (glycosylation sequences), etc.

In any case, the functional variant will retain the catalytic activity of the enzyme of which it is a variant to the degree that the functional variant exhibits at least about 50 percent, and preferably about 60%, 70%, 80%, 90%, 100% or more, of the activity of the enzyme of which it is a variant, when assayed under standard conditions as recognized by those of skill in the art.

Further, the present invention also includes nucleic acid sequences encoding enzymes and functional variants of enzymes utilized in the present invention. The nucleic acid sequences may be deooxyribonucleotides, ribonucleotides, or modified forms of either, and may be single- or double-stranded. The nucleic acid sequences of the present invention include any that are listed herein, and are also intended to encompass variants thereof. For example, variants of the nucleic acids may not be identical to the listed sequence but may still encode an identical amino acid sequence due to the redundancy of the genetic code. Alternatively, some changes in the sequences of the present invention may be made (e.g. substitutions, deletions or additions) that result in changes in the encoded amino acid sequence, so long as the encoded amino acid sequence is a functional variant of the amino acid sequences of the present invention as described above. Examples include but are not limited to changes that cause conservative amino acid substitutions in the enzyme; and changes that result in non-conservative substitutions, or deletion or additions in the amino acid sequences. Such changes may be introduced for any reason, e.g. in order to alter post-translational modifications of the enzyme; to increase or decrease solubility; to prevent or introduce steric interactions in the translated polypeptide, etc. In general, variants of the nucleic acid sequences of the present invention will exhibit at least about 50 percent, and preferably about 60%, 70%, 80%, 90%, 95%, or 100% homology to the sequences of the present invention, as determined by comparative procedures that are well known to those of skill in the art. Such variants are also characterized by exhibiting the ability to bind to the sequences of the present invention under conditions of high stringency. High stringency binding assays are well-known to those of skill in the art and can readily be applied to test potential variants of sequences of the present invention.

Sequence homologies as described herein are not intended to refer to nucleic acid sequences encoding heterologous amino acid sequences that are derived from sources other than the reference amino acid sequence, and which are attached to or included in the polypeptide sequence of an enzyme for various other purposes. For example, such nucleic acid sequences may encode heterologous amino acid sequences including but not limited to sequences that facilitate polypeptide isolation (e.g. Histidine tags), sequences that facilitate secretion or localization of the polypeptide within the cell, (e.g., various leader sequences), and sequences that code for glycosylation sites (glycosylation sequences), etc.

Other variations of the nucleic acid sequences of the present invention that are intended to be encompassed by the present invention are sequences which have been altered for convenience or improvement in genetic engineering of the nucleic acid sequences, or the expression of the amino acid sequences they encode. In general, such alterations will not affect the sequence of the polypeptide that is ultimately translated from the nucleic acid sequence; or the polypeptide will still fulfill the criteria set forth above for a functional variant. Examples of this type of alteration include but are not limited to: the inclusion of convenient restriction endonuclease sites in a nucleic acid sequence to facilitate manipulation of the sequence (e.g. for insertion of a sequence into a vector); the inclusion, deletion, or other change in a sequence or sequences involved in expression of the amino acid sequence (e.g. inclusion of any of various promoter and/or enhancer sequences, stop signals, super promoters, and various other sequences that modify expression of the nucleic acid sequence); the inclusion of sequences that facilitate interaction of a vector with the nucleic acid of a host organism; etc.

Further, the nucleic acid sequences of the present invention may be chemically modified or include non-traditional bases for any of many reasons that are well-known to those of skill in the art, for example, to promote stability of the nucleic acid, or to confer a desired steric conformation.

The Sod enzymes utilized in the present invention are not secreted by the Mycobacterium into which they have been introduced by genetic manipulation. Such Sod enzymes may be of a type that is naturally not secreted. Alternatively, such Sod enzymes may be of a type that is naturally secreted by its native host, but that has been genetically engineered to lose the capability of being secreted and is thus cytosol-bound.

The present invention also provides vaccine preparations for use in eliciting an immune response against tuberculosis. The vaccine preparations include at least one rBCG strain as described herein, and a pharmacologically suitable carrier. The preparation of such compositions for use as vaccines is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients that are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of rBCG bacteria in the formulations may vary. However, in general, the amount in the formulations will be from about 1-99 percent by weight or by volume. The vaccine preparations of the present invention may further comprise an adjuvant, suitable examples of which include but are not limited to Seppic, Quil A, Alhydrogel, etc. Further, the vaccine preparations of the present invention may contain a single type of rBCG. Alternatively, more than one type of rBCG may be utilized in a vaccine.

The present invention also provides method of eliciting an immune response to tuberculosis and methods of vaccinating a mammal against tuberculosis. By eliciting an immune response, we mean that administration of the vaccine preparation of the present invention causes the synthesis of specific antibodies (at a titer in the range of 1 to 1×10⁶, preferably 1×10³, more preferable in the range of about 1×10³ to about 1×10⁶, and most preferably greater than 1×10⁶) and/or cellular proliferation, as measured, e.g. by ³H thymidine incorporation. The methods involve administering a composition comprising a rBCG strain of the present invention in a pharmacologically acceptable carrier to a mammal. The vaccine preparations of the present invention may be administered by any of the many suitable means which are well known to those of skill in the art, including but not limited to by injection, orally, intranasally, by ingestion of a food product containing the rBCG, etc. In preferred embodiments, the mode of administration is subcutaneous or intramuscular.

Such strains will not only be useful in providing protection against Tuberculosis by themselves or as part of prime boost regimens but will also be useful for delivery of transgenes against other diseases as vaccines against other diseases by themselves or part of prime boost regimens. That is, this invention has utility in the development of vaccine antigen delivery vectors. A Mycobacterium vector is defined herein as any Mycobacterium strain engineered to express at least one passenger nucleotide sequence (herein referred to as “PNS”) comprised of DNA or RNA and encoding any combination of antigens, immunoregulatory factors or adjuvants, as set forth below. The PNS can be introduced into the chromosome or as part of an expression vector using compositions and methods well known in the art (Jacobs et al., Nature 327:532-535; 1987; Barletta et al., Res Microbiol. 141:931-939; 1990; Kawahara et al., Clin Immunol. 105:326-331; 2002; Lim et al., AIDS Res Hum Retroviruses. 13:1573-1581; 1997; Chujoh et al., Vaccine, 20:797-804; 2001; Matsumoto et al., Vaccine, 14:54-60; 1996; Haeseleer et al., Mol Biochem Parasitol., 57:117-126; 1993). In the present invention, the Mycobacterium vector may carry a PNS encoding an immunogen, which may be either a foreign immunogen from viral, bacterial and parasitic pathogens, or an endogenous immunogen, such as but not limited to an autoimmune antigen or a tumor antigen. The immunogens may be the full-length native protein, chimeric fusions between the foreign immunogen and an endogenous protein or mimetic, a fragment or fragments thereof of an immunogen that originates from viral, bacterial and parasitic pathogens.

As used herein, “foreign immunogen” means a protein or fragment thereof, which is not normally expressed in the recipient animal cell or tissue, such as, but not limited to, viral proteins, bacterial proteins, parasite proteins, cytokines, chemokines, immunoregulatory agents, or therapeutic agents.

An “endogenous immunogen” means a protein or part thereof that is naturally present in the recipient animal cell or tissue, such as, but not limited to, an endogenous cellular protein, an immunoregulatory agent, or a therapeutic agent. Alternatively or additionally, the immunogen may be encoded by a synthetic gene and may be constructed using conventional recombinant DNA methods known to those of skill in the art.

The foreign immunogen can be any molecule that is expressed by any viral, bacterial, or parasitic pathogen prior to or during entry into, colonization of, or replication in their animal host; the Mycobacterium vector may express immunogens or parts thereof that originate from viral, bacterial and parasitic pathogens. These pathogens can be infectious in humans, domestic animals or wild animal hosts.

The viral pathogens, from which the viral antigens are derived, include, but are not limited to, Orthomyxoviruses, such as influenza virus (Taxonomy ID: 59771; Retroviruses, such as RSV, HTLV-1 (Taxonomy ID: 39015), and HTLV-II (Taxonomy ID: 11909), Herpes viruses such as EBV Taxonomy ID: 10295); CMV (Taxonomy ID: 10358) or herpes simplex virus (ATCC #: VR-1487); Lentiviruses, such as HIV-1 (Taxonomy ID: 12721) and HIV-2 Taxonomy ID: 11709); Rhabdoviruses, such as rabies; Picornoviruses, such as Poliovirus (Taxonomy ID: 12080); Poxviruses, such as vaccinia (Taxonomy ID: 10245); Rotavirus (Taxonomy ID: 10912); and Parvoviruses, such as adeno-associated virus 1 (Taxonomy ID: 85106).

Examples of viral antigens can be found in the group including but not limited to the human immunodeficiency virus antigens Nef (National Institute of Allergy and Infectious Disease HIV Repository Cat. # 183; Genbank accession # AF238278), Gag, Env (National Institute of Allergy and Infectious Disease HIV Repository Cat. # 2433; Genbank accession # U39362), Tat (National Institute of Allergy and Infectious Disease HIV Repository Cat. # 827; Genbank accession # M13137), mutant derivatives of Tat, such as Tat-Δ31-45 (Agwale et al., Proc. Natl. Acad. Sci. In press. Jul. 8^{th}; 2002), Rev (National Institute of Allergy and Infectious Disease HIV Repository Cat. # 2088; Genbank accession # L14572), and Pol (National Institute of Allergy and Infectious Disease HIV Repository Cat. # 238; Genbank accession # AJ237568) and T and B cell epitopes of gp120 (Hanke and McMichael, AIDS Immunol Lett., 66:177; 1999); (Hanke, et al., Vaccine, 17:589; 1999); (Palker et al., J. Immunol., 142:3612-3619; 1989) chimeric derivatives of HIV-1 Env and gp120, such as but not restricted to fusion between gp120 and CD4 (Fouts et al., J. Virol., 74:11427-11436; 2000); truncated or modified derivatives of HIV-1 env, such as but not restricted to gp140 (Stamatos et al., J Virol, 72:9656-9667; 1998) or derivatives of HIV-1 Env and/or gp140 thereof (Binley, et al., J Virol, 76:2606-2616_{—}; 2002); (Sanders, et al., J_{—}Virol, 74:5091-5100; 2000); (Binley, et al., J Virol, 74:627-643; 2000), the hepatitis B surface antigen (Genbank accession # AF043578); (Wu et al., Proc. Natl. Acad. Sci., USA, 86:4726-4730; 1989); rotavirus antigens, such as VP4 (Genbank accession # AJ293721; Mackow et al., Proc. Natl. Acad. Sci., USA, 87:518-522; 1990) and VP7 (Genbank accession # AY003871;) (Green et al., J. Virol., 62:1819-1823; 1988), influenza virus antigens such as hemagglutinin or (Genbank accession # AJ404627); (Pertmer and Robinson, Virology, 257:406; 1999); nucleoprotein (Genbank accession # AJ289872); (Lin et al., Proc. Natl. Acad. Sci., 97: 9654-9658; 2000) herpes simplex virus antigens such as thymidine kinase (Genbank accession # AB047378); (Whitley et al., New Generation Vaccines, 825-854; 2004).

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

Examples of protective antigens of bacterial pathogens include the somatic antigens of enterotoxigenic E. coli, such as the CFA/I fimbrial antigen (Yamamoto et al., Infect. Immun., 50:925-928; 1985) and the nontoxic B-subunit of the heat-labile toxin (Klipstein et al., Infect. Immun., 40:888-893; 1983); pertactin of Bordetella pertussis (Roberts et al., Vacc., 10:43-48; 1992), adenylate cyclase-hemolysin of B. pertussis (Guiso et al., Micro. Path., 11:423-431; 1991), fragment C of tetanus toxin of Clostridium tetani (Fairweather et al., Infect. Immun., 58:1323-1326; 1990), OspA of Borellia burgdorferi (Sikand, et al., Pediatrics, 108:123-128; 2001); (Wallich, et al., Infect Immun, 69:2130-2136; 2001), protective paracrystalline-surface-layer proteins of Rickettsia prowazekii and Rickettsia typhi (Carl, et al., Proc Natl Acad Sci USA, 87:8237-8241; 1990), the listeriolysin (also known as “Llo” and “Hly”) and/or the superoxide dismutase (also know as “SOD” and “p60”) of Listeria monocytogenes (Hess, et al., Infect. Immun. 65:1286-92; 1997; (Hess, et al., Proc. Natl. Acad. Sci. 93:1458-1463; 1996); (Bouwer, et al., J. Exp. Med. 175:1467-71;_{—}1992), the urease of Helicobacter pylori (Gomez-Duarte, et al., Vaccine 16, 460-71; 1998); Corthesy-Theulaz, et al., Infection & Immunity 66, 581-6; 1998), and the receptor-binding domain of lethal toxin and/or the protective antigen of Bacillus anthrax (Price, et al., Infect. Immun. 69, 4509-4515; 2001).

The parasitic pathogens, from which the parasitic antigens are derived, include but are not limited to, Plasmodium spp., such as Plasmodium falciparum (ATCC#: 30145); Trypanosome spp., such as Trypanosoma cruzi (ATCC#: 50797); Giardia spp., such as Giardia intestinalis (ATCC#: 30888D); Boophilus spp., Babesia spp., such as Babesia microti (ATCC#: 30221); Entamoeba spp., such as Entamoeba histolytica (ATCC#: 30015); Eimeria spp., such as Eimeria maxima (ATCC# 40357); Leishmania spp. (Taxonomy ID: 38568); Schistosome spp., Brugia spp., Fascida spp., Dirofilaria spp., Wuchereria spp., and Onchocerea spp.

Examples of protective antigens of parasitic pathogens include the circumsporozoite antigens of Plasmodium spp. (Sadoffet al., Science 240:336-337; 1988), such as the circumsporozoite antigen of P. bergerii or the circumsporozoite antigen of P. falciparum; the merozoite surface antigen of Plasmodium spp. (Spetzler et al., Int. J. Pept. Prot. Res., 43:351-358; 1994); the galactose specific lectin of Entamoeba histolytica (Mann et al., Proc. Natl. Acad. Sci., USA, 88:3248-3252; 1991), gp63 of Leishmania spp. (Russell et al., J. Immunol., 140:1274-1278; 1988); (Xu and Liew, Immunol., 84: 173-176; 1995), gp46 of Leishmania major (Handman et al., Vaccine, 18: 3011-3017; 2000), paramyosin of Brugia malayi (Li et al., Mol. Biochem. Parasitol., 49:315-323; 1991), the triose-phosphate isomerase of Schistosoma mansoni (Shoemaker et al., Proc. Natl. Acad. Sci., USA, 89:1842-1846; 1992); the secreted globin-like protein of Trichostrongylus colubriformis (Frenkel et al., Mol. Biochem. Parasitol., 50:27-36; 1992); the glutathione-S-transferase's of Frasciola hepatica (Hillyer et al., Exp. Parasitol., 75:176-186; 1992), Schistosoma bovis and S. japonicum (Bashir et al., Trop. Geog. Med., 46:255-258; 1994); and KLH of Schistosoma bovis and S. japonicum (Bashir et al., supra, 1994).

As mentioned earlier, the Mycobacterium vector may carry a PNS encoding an endogenous immunogen, which may be any cellular protein, immunoregulatory agent, or therapeutic agent, or parts thereof, that may be expressed in the recipient cell, including but not limited to tumor, transplantation, and autoimmune immunogens, or fragments and derivatives of tumor, transplantation, and autoimmune immunogens thereof. Thus, in the present invention, Mycobacterium vector may carry a PNS encoding tumor, transplant, or autoimmune immunogens, or parts or derivatives thereof. Alternatively, the Mycobacterium vector may carry synthetic PNS's (as described above), which encode tumor-specific, transplant, or autoimmune antigens or parts thereof.

Examples of tumor specific antigens include prostate specific antigen (Gattuso et al., Human Pathol., 26:123-126; 1995), TAG-72 and CEA (Guadagni et al., Int. J. Biol. Markers, 9:53-60; 1994), MAGE-1 and tyrosinase (Coulie et al., J. Immunothera., 14:104-109; 1993). Recently, it has been shown in mice that immunization with non-malignant cells expressing a tumor antigen provides a vaccine effect, and also helps the animal mount an immune response to clear malignant tumor cells displaying the same antigen (Koeppen et al., Anal. N.Y. Acad. Sci., 690:244-255; 1993).

Examples of transplant antigens include the CD3 molecule on T cells (Alegre et al., Digest. Dis. Sci., 40:58-64; 1995). Treatment with an antibody to CD3 receptor has been shown to rapidly clear circulating T cells and reverse cell-mediated transplant rejection (Alegre et al., supra, 1995).

Examples of autoimmune antigens include IAS β chain (Topham et al., Proc. Natl. Acad. Sci., USA, 91:8005-8009; 1994). Vaccination of mice with an 18 amino acid peptide from IAS β chain has been demonstrated to provide protection and treatment to mice with experimental autoimmune encephalomyelitis (Topham et al., supra, 1994).

Mycobacterium Vectors which Express an Adjuvant

It is feasible to construct Mycobacterium vectors that carry PNS encoding an immunogen and an adjuvant, and are useful in eliciting augmented host responses to the vector and PNS-encoded immunogen. Alternatively, it is feasible to construct Mycobacterium vectors that carry PNS encoding an adjuvant, which are administered in mixtures with other Mycobacterium vectors that carry PNS encoding at least one immunogen to increase host responses to said immunogen encoded by the partner Mycobacterium vector.

The particular adjuvant encoded by PNS inserted in said Mycobacterium vector is not critical to the present invention and may be the A subunit of cholera toxin (i.e. CtxA; Genbank accession no. X00171, AF175708, D30053, D30052), or parts and/or mutant derivatives thereof (E.g. the A1 domain of the A subunit of Ctx (i.e. CtxA1; Genbank accession no. K02679)), from any classical Vibrio cholerae (E.g. V. cholerae strain 395, ATCC # 39541) or El Tor V. cholerae (E.g. V. cholerae strain 2125, ATCC # 39050) strain. Alternatively, any bacterial toxin that is a member of the family of bacterial adenosine diphosphate-ribosylating exotoxins (Krueger and Barbier, Clin. Microbiol. Rev., 8:34; 1995), may be used in place of CtxA, for example the A subunit of heat-labile toxin (referred to herein as EltA) of enterotoxigenic Escherichia coli (Genbank accession # M35581), pertussis toxin S1 subunit (E.g. ptxS1, Genbank accession # AJ007364, AJ007363, AJ006159, AJ006157, etc.); as a further alternative the adjuvant may be one of the adenylate cyclase-hemolysins of Bordetella pertussis (ATCC # 8467), Bordetella bronchiseptica (ATCC # 7773) or Bordetella parapertussis (ATCC # 15237), E.g. the cyaA genes of B. pertussis (Genbank accession no. X14199), B. parapertussis (Genbank accession no. AJ249835) or B. bronchiseptica (Genbank accession no. Z37112).

Mycobacterium Vector which Express an Immunoregulatory Agent

Yet another approach entails the use of Mycobacterium vector that carry at least one PNS encoding an immunogen and a cytokine, which are used to elicit augmented host responses to the PNS-encoded immunogen Mycobacterium vector. Alternatively, it is possible to construct a Mycobacterium vector that carries a PNS encoding said cytokine alone, which are used in admixtures with at least one other Mycobacterium vector carrying a PNS encoding an immunogen to increase host responses to PNS-encoded immunogens expressed by the partner Mycobacterium vector.

The particular cytokine encoded by the Mycobacterium vector is not critical to the present invention includes, but not limited to, interleukin-4 (herein referred to as “IL-4”; Genbank accession no. AF352783 (Murine IL4) or NM_{—}000589 (Human IL-4)), IL-5 (Genbank accession no. NM_{—}010558 (Murine IL-5) or NM_{—}000879 (Human IL-5)), IL-6 (Genbank accession no. M20572 (Murine IL-6) or M29150 (Human IL-6)), IL-10 (Genbank accession no. NM_{—}010548 (Murine IL-10) or AF418271 (Human IL-10)), Il-12p40 (Genbank accession no. NM_{—}008352 (Murine IL-12 p40) or AY008847 (Human IL-12 p40)), IL-12ₚ₇₀ (Genbank accession no. NM_{—}008351/NM_{—}008352 (Murine IL-12 p35/40) or AF093065/AY008847 (Human IL-12 p35/40)), TGFβ (Genbank accession no. NM_{—}011577 (Murine TGFβ1) or M60316 (Human TGFβ1)), and TNFα Genbank accession no. X02611 (Murine TNFα) or M26331 (Human TNFα)).

In yet a further embodiment, such Mycobacterium which express modified Sod enzyme when used by themselves or in prime boost regimens as vaccines against Tuberculosis or other diseases can further enhance immunity to said Mycobacterium strain or to said antigen encoded by PNS by expression of transgenes that permit escape of the organism from the endosome, that promote apoptosis or are cytokines with inherent aduvant activity. In another preferred embodiment of the present invention, the two-component TB vaccine can include attenuated Mycobacterium strains that carry a PNS encoding an endosomalytic proteins, such as Listeriolysin (Genbank accession no. CAA59919 or CAA42639), Hemolysin (Genbank accession no AAC24352 or CAA0535) and Perfringolysin (Genbank accession no. P19995 or AAA23271), which imparts the ability to degrade the endosome, either partially resulting in leakage of antigens into the cytoplasm or to the extent that the endosome is ruptured and said Mycobacterium strain escapes this compartment and resides in the cytoplasm (Hess et al., Proc Natl Acad. Sci., 95:5299-5304; 1998; Grode et al., Clin Invest., 115:2472-2479; 2005).

Such strains will not only be useful as vaccine strains against Tuberculosis and other diseases but will also provide anticancer activity when applied locally to tumors such as bladder cancer using procedures well know in the art (Silverstein et al., JAMA., 229:688; 1974; Morales et al., J Urol., 116:180-183; 1976; Martinez et al., Eur Urol., 3:11-22; 1977).

The following examples are to be considered as exemplary of various aspects of the present invention and are no intended to be limiting with respect to the practice of the invention. Those of ordinary skill in the art will appreciate that alternative materials, conditions, and procedures may be varied and remain within the skill of the ordinarily skilled artisan without departing from the general scope of the invention as taught in the specification.

### EXAMPLES

### Methods

General Molecular Biology Techniques

Restriction endonucleases (herein “REs”); New England Biolabs Beverly, Mass.), T4 DNA ligase (New England Biolabs, Beverly, Mass.) and Taq polymerase (Life Technologies, Gaithersburg, Md.) are used according to the manufacturers' protocols; Plasmid DNA is prepared using small-scale (Qiagen Miniprep^{R} kit, Santa Clarita, Calif.) or large-scale (Qiagen Maxiprep^{R} kit, Santa Clarita, Calif.) plasmids DNA purification kits according to the manufacturer's protocols (Qiagen, Santa Clarita, Calif.); Nuclease-free, molecular biology grade milli-Q water, Tris-HCl (pH 7.5), EDTA pH 8.0, 1M MgCl₂, 100% (v/v) ethanol, ultra-pure agarose, and agarose gel electrophoresis buffer are purchased from Life Technologies, Gaithersburg, Md. RE digestions, PCRs, DNA ligation reactions and agarose gel electrophoresis is conducted according to well-known procedures (Sambrook, et al., Molecular Cloning: A Laboratory Manual. 1, 2, 3; 1989; Straus et al., Proc Natl Acad Sci USA. March; 87(5): 1889-93; 1990). Nucleotide sequencing to verify the DNA sequence of each recombinant plasmid described in the following sections was accomplished by conventional automated DNA sequencing techniques using an Applied Biosystems automated sequencer, model 373A.

PCR primers are purchased from commercial vendors such as Sigma (St. Louis, Mo.) and are synthesized using an Applied Biosystems DNA synthesizer (model 373A). PCR primers are used at a concentration of 150-250 μM and annealing temperatures for the PCR reactions are determined using Clone manager software version 4.1 (Scientific and Educational Software Inc., Durham N.C.). PCRs are conducted in a Strategene Robocycler, model 400880 (Strategene, La Jolla, Calif.). The PCR primers for the amplifications are designed using Clone Manager® software version 4.1 (Scientific and Educational Software Inc., Durham N.C.). This software enabled the design PCR primers and identifies RE sites that are compatible with the specific DNA fragments being manipulated. PCRs are conducted in a thermocycler device, such as the Strategene Robocycler, model 400880 (Strategene), and primer annealing, elongation and denaturation times in the PCRs are set according to standard procedures (Straus et al., supra, 1990). The RE digestions and the PCRs are subsequently analyzed by agarose gel electrophoresis using standard procedures (Straus et al., supra, 1990; and Sambrook et al., supra, 1989). A positive clone is defined as one that displays the appropriate RE pattern and/or PCR pattern. Plasmids identified through this procedure can be further evaluated using standard DNA sequencing procedures, as described above.

Bacterial strains and their cultivation. Escherichia coli strains, such as DH5α and Sable2^{R}, are purchased from Life Technologies (Bethesda, Md.) and serve as initial host of the recombinant plasmids described in the examples below. Recombinant plasmids are introduced into E. coli strains by electroporation using an high-voltage eletropulse device, such as the Gene Pulser (BioRad Laboratories, Hercules, Calif.)), set at 100-200Ω, 15-25 μF and 1.0-2.5 kV, as described (Straus et al., supra, 1990). Optimal electroporation conditions are identified by determining settings that result in maximum transformation rates per mcg DNA per bacterium.

Bacterial strains are grown on tryptic soy agar (Difco, Detroit, Mich.) or in tryptic soy broth (Difco, Detroit, Mich.), which are made according to the manufacturer's directions. Unless stated otherwise, all bacteria are grown at 37° C. in 5% (v/v) CO₂ with gentle agitation. When appropriate, the media are supplemented with antibiotics (Sigma, St. Louis, Mo.). Bacterial strains are stored at −80° C. suspended in (Difco) containing 30% (v/v) glycerol (Sigma, St. Louis, Mo.) at ca. 10⁹ colony-forming units (herein referred to as “cfu”) per ml.

Allelic Exchange in BCG

The prior art teaches methods for introducing altered alleles into Mycobacterium strains and those skilled in the art will be capable of interpreting and executing said methods (Parish et al., Microbiology, 146: 1969-1975; 2000). A novel method to generate an allelic exchange plasmid entails the use of synthetic DNA. The advantage of this approach is that the plasmid product will have a highly defined history and will be compliant with federal regulations, whereas previously used methods, although effective, have poorly documented laboratory culture records and thus are unlikely to be compliant with federal regulations. Compliance with federal regulations is essential if a product is to be licensed for use in humans by United States and European regulatory authorities.

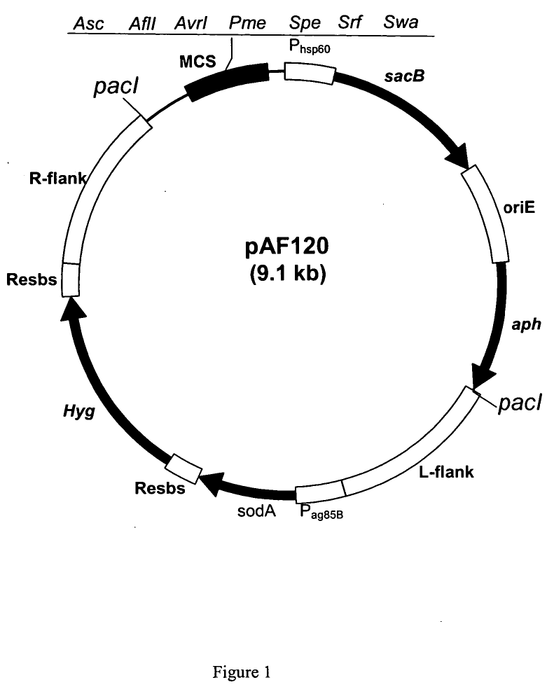

A suicide vector for allelic exchange in Mycobacterium is a plasmid that has the ability to replicate in E. coli strains but is incapable of replication in Mycobacterium spp., such as M. tb and BCG. The specific suicide vector for use in allelic exchange procedures in the current invention is not important and can be selected from those available from academic (Pavelka et al., J Bacteriol. 181(16): 4780-9; 1999) and commercial sources. A preferred design of a suicide plasmid for allelic exchange is shown in FIG. 1. The plasmid is comprised of the following DNA segments: an oriE sequence for the plasmid to replicate in E. coli (Genebank accession # L09137), a kanamycin-resistant sequence for selection in both E. coli and Mycobacterium (Genebank accession # AAM97345), a levansucrase encoding gene (SacB) (gene bank accession number: Y489048), which confers the bacteria sensitivity to sucrose; a gene encoding Listeria monocytogenes EGD-e Superoxide dismutase (accession number: NT01LM1550) flanked by left and a right flank region of the sodA gene to be replaced. In addition, the plasmid also contains a hygromycin-resistance gene (bank accession number DQ005458), which is used to confer a selectable phenotype for the convenience of selecting the resultant mutant. The hygromycin resistance gene is flanked by the resolvase binding sequence (gene bank accession number: X03526), which will serve as the binding site for the resolvase to remove the hygromycin resistance gene from the construct at the end (Bardarov et al., Microbiology 148, 3007-3017, 2002).

Construction of such a suicide vectors can be accomplished using standard recombinant DNA techniques as described above. However, current regulatory standards have raised the specter of introducing prion particles acquired from products exposed to bovine products containing BSE-infected material. To avoid introducing materials (e.g. DNA sequences) into the target strain of unknown origin, it is preferable that all DNA in the suicide vector are made synthetically by commercial sources (e.g. Picoscript, Inc). Accordingly, a preferred method for constructing suicide vectors is to assemble a plan of the DNA sequences using DNA software (e.g. Clone Manager), then to synthesize the DNA on a fee-for-service basis by any commercial supplier that offer such a service (e.g. Picoscript Inc.). The suicide vector depicted schematically in FIG. 1 was obtained in this manner.

The configuration of the suicide vector described above has advantages, as this plasmid contains two antibiotic selection markers, thus minimizing selection of spontaneous mutants that display resistance to one antibiotic, which occurs at ca. 1/10⁸ per generation. Spontaneous resistance to two antibiotics is extremely rare and only occurs at ca. 1/10¹⁶ per generation. Thus, there is less that 1/10⁶ probability of double resistant strains emerging in the cultures used to execute the allelic exchange procedure.

The process of allele exchange is illustrated schematically in FIG. 2, which outlines the major steps of the procedure. Those steps are described in detail below: BCG Danish 1331 was cultured in 7H9 medium with 10% of OADC (oleic acid-albumin-dextrose-catalase) (BD Gibco) and 0.05% (v/v) of Tyloxapol (research and diagnostic lab) supplementation. When the culture reached log phase, the bacteria were collected and prepared as described previously (Sun et al., 2004) for electroporation. Five micrograms of the allele exchange plasmid is introduced into freshly prepared electrocompetent cells using standard methodologies. After electroporation, the cells are cultured overnight in 7H9 medium with 10% (v/v) OADC and 0.05% (v/v) of Tyloxapol supplementation. Then the cells were plated on 7H10 plates containing 50 ug/ml of both kanamycin and hygromycin. The resultant colonies are picked and cultured in 7H9 medium containing 10% (v/v) of sucrose for negative selection during allelic exchange process to enrich cultures with strains that have undergone the final DNA recombination step and completed the allelic exchange. The obtained culture is plated on 7H10 plates to obtain individual colonies. The resultant colonies are screened first for the phenotype of resistance to hygromycin but sensitive for kanamycin. Then presence of Listeria soda gene in place of mycobacterial soda gene is confirmed by PCR to amplify the chromosome region followed by sequencing analysis of the PCR product. A flow chart outlining the main steps of this procedure is given in FIG. 2, and Table 1 describes the suicide vector, pAF 120, which is also depicted in FIG. 1.

TABLE 1
Suicide vector used in the invention

| Name | Backbone | Specific allele for allele exchange |
|---|---|---|
| pAF120 | pAF 100 | Listeria sodA gene flanked by 1 kb flanks |
| | | of the native sodA gene |

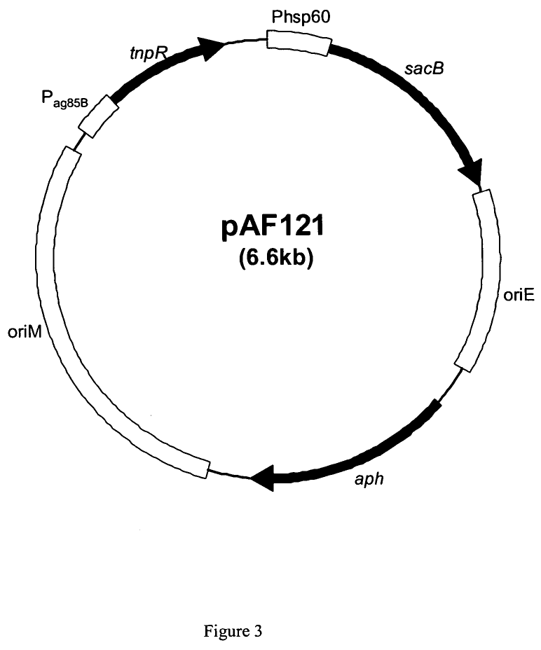

Once the strain with the desired recombination is obtained, the bacteria are amplified as above for the second round of electroporation to remove the hygromycin gene from the chromosome. This is accomplished by introducing a helper plasmid that encodes transposon gamma-delta resolvase (tnpr) (gene bank accession number: J01844), which is able to cut the hygromycin gene from the chromosome. The design of the helper plasmid pAF121 is shown in FIG. 3. Briefly, it contains the origin of replication in mycobacterium (gene bank accession number: M23557) for its ability to replicate in mycobacterium; an antibiotic selection marker (i.e. kanamycin, Genebank accession # AAM97345) to select for the presence of the plasmid in the cells and the sacB gene as described above for the down stream negative selection to deselect the plasmid. The plasmid is introduced into the cells by standard electroporation. Then the bacteria are cultured in the medium containing kanamycin to select for the strains harboring the helper plasmid. Then the bacteria are expanded briefly followed by inoculated on the solid medium to isolate the individual colonies. The resultant colonies are screened for its sensitivity for hygromycin and resistance to kanamycin. Once the mutant with the desired phenotype is obtained, it will be cultured in the medium with containing 10% sucrose for the final step of deselect the helper plasmid.

Formulation and Vaccination Strategies.

The strategies for vaccine formulation structured on studies to determine maximum viability and stability throughout the manufacturing process. This includes determination maximum organism viability (live to dead) during culture utilizing a variety of commonly used medium for the culture of Mycobacteria to include the addition of glycerol, sugars, amino acids, and detergents or salts. After culture cells are harvested by centrifugation or tangential flow filtration and resuspended in a stabilizing medium that allows for protection of cells during freezing or freeze-drying process. Commonly used stabilizing agents include sodium glutamate, or amino acid or amino acid derivatives, glycerol, sugars or commonly used salts. The final formulation will provide sufficient viable organism to be delivered by intradermal, percutaneous injection, perfusion or oral delivery with sufficient stability to maintain and adequate shelf-life for distribution and use.

Preclinical Evaluation of TB Vaccines

General safety test. BALB/c mice in groups of six are infected intraperitoneally with 2×10⁶ CFU of the rBCG strain(s) of interest and the analogous parental strains. The animals are monitored for general health and body weight for 14 days post infection. Animals that receive the BCG and rBCG strains should remain healthy, and should neither lose weight nor display overt signs of disease during the observation period.

Virulence of novel rBCG strains in immunocompetent mice. Groups of 15 immunocompetent BALB/c mice are infected intravenously with 2×10⁶ rBCG and BCG parental strain respectively. At day 1 post infection, three mice in each group are sacrificed and CFU in spleen, lung and live are analyzed to ensure each animal has equal infection dose. At week 4, 8, 12, and 16 post infection, three mice in each group are sacrificed and CFU in spleen, liver and lung is obtained to assess the in vivo growth of the rBCG strains as compared to the parental BCG strain. Positive results are demonstrated by rBCG strains displaying similar virulence to that of the parental BCG.

Stringent safety test in immunocompromised mice. Immunocompromised mice possessing the SCID (severe combined immunodeficiency) in groups of 10 are infected intravenously with 2×10⁶ cfu rBCG and the parental BCG strain respectively. The first day after infection, three mice in each group are sacrificed and cfu in spleen, liver and lung is assessed to verify the inoculation doses. The remaining seven mice in each group are monitored for general health and body weight. The survival of these mice is followed and positive results are indicated by the survival of rBCG-infected mice being no worse than the parental strain infected animal during the entire observation period

Guinea pig safety test. The safety of rBCG strains is also assessed in the guinea pig model in comparison to the parental BCG vaccine, which has a well-established safety profile in humans. First, the effect of the vaccine on the general health status of the animals, including weight gain, is examined. Guinea pigs are immunized intramuscularly with 10⁷ (100× of vaccination dose) cfu of the recombinant and parental strains, and the animals are monitored for general health and body weight for six weeks. Post mortem examination is performed for animals that die before the six weeks period. All animals are sacrificed at the end of 6 weeks post infection and gross pathology is performed. Positive results are indicated when there is no body weight loss, no abnormal behavior and all organs appear normal at 6 weeks necropsy, and when no adverse health effects are observed for rBCG-Pfo vaccine, and experimental animals gain weight at the normal rate compared to the parental strain inoculated animals.

At the same time, bacterial levels in animal organs are monitored. Guinea pigs immunized with either the parental or recombinant vaccine are euthanized at various intervals after inoculation, after which the lungs, spleens, and regional (inguinal) lymph nodes are assayed for CFU of BCG or rBCG.

Toxicity test. To evaluate the toxicity of the rBCG strains, guinea pigs (12 in each group) are vaccinated intradermally with one dose, four times higher than the single dose or four times lower than the single dose of human use rBCG strains, BCG parental strain or saline respectively. At days 3 post vaccination, six animals are sacrificed to access the acute effects of the vaccine on these animals. At day 28 days post vaccination, the remaining six animals are sacrificed to evaluate the chronic effects of on the animals. At both time points, the body weight of each animal is obtained, and gross pathology and appearance of the injection sites are examined. Blood is taken for blood chemistry, and the histopathology of the internal organs and injection sites are performed.

Murine protection study. C57B1/6 mice (female, 5-6 weeks of age) in groups of 13 are immunized subcutaneously with 10⁶ CFU of rBCG, parental BCG or saline. Another group of mice is used as healthy controls. Eight weeks after immunization, mice are challenged with M. tb Erdman strain (or H37Rv Kan-resistant strain) by an aerosol generated from a 10-ml single-cell suspension containing a total of 10⁷ CFU of the challenge strain, a dose that delivers 100 live bacteria to the lungs of each animal, as described previously and monitored for survival along with unchallenged animals. Following the challenge, the animals are monitored for weight loss and general health. The first day after challenge, three mice in each group are sacrificed for lung cfu to confirm challenge dose and one is sacrificed for spleen and lung histopathology. Then five weeks after challenge, nine animals in each group are sacrificed, and histopathology and microbiology analysis of the animal are performed. Lung and spleen tissues from six mice are evaluated for cfu counts (plates with selection supplements are used to distinguish vaccine strain from challenge strain). If challenged with H37Rv-kan resistant strain, Kan or TCH (thiophene-2-carboxylic acid hydrazide) are used to distinguish the challenge strain from the vaccine strain. If the M. tb Erdman strain is used to challenge, TCH is used to distinguish the vaccine strain from the challenge strain (BCG is susceptible, but M. tb is naturally resistant).

Induction of cutaneous delayed-type hypersensitivity (DTH). Specific pathogen free (SPF) guinea pigs are immunized intradermally with 10³ rBCG or BCG parental strains. Nine weeks after immunization, the animals are shaved over the back and injected intradermally with 10 μg of PPD (protein purified derivative) in 100 μl of phosphate buffered saline. After 24 hs, the diameter of hard induration is measured. rBCG strains induce the DTH at a level equal to or greater than that induced by parental BCG strains.

Guinea pig challenge study. To determine the efficacy of the rBCG vaccines against M. tb challenge, guinea pigs are immunized (young adult SPF Hartley, 250-300 grams, male) in groups of 12, each with rBCG, parental BCG strain or saline. The vaccines and controls are administered intradermally with 10⁶ cfu. At 10 weeks after immunization, the rBCG-, BCG- and sham-immunized animals are challenged by aerosol with the M. tb by an aerosol generated from a 10-ml single-cell suspension containing a total of 10⁷ cfu of M. tb; this procedure delivers ˜100 live bacteria to the lungs of each animal, as described previously (Brodin et al., J Infect Dis. 190(1): 2004). Following challenge, the animals are monitored for survival along with a healthy group of unvaccinated, unchallenged animals. Following the challenge, the animals are monitored for weight loss and general health. Six animals in each group are sacrificed at 10 weeks post challenge and the remaining six in each group at 70 weeks post challenge for long term evaluation. At both time points, histopathology and microbiology analysis of the animal are performed. Lung and spleen tissues are evaluated for histopathology and cfu count (plates with selection supplements are used to distinguish vaccine strain from challenge strain). If challenged with the H37Rv-kan resistant strain, Kan or TCH are used to distinguish challenge strain from the vaccine strain; if M. tb Erdman strain is used to challenge, TCH is used to distinguish the vaccine strain from the challenge strain (BCG is susceptible but M. tb is naturally resistant). Sham immunized animals die most rapidly after challenge, whereas the rBCG-immunized animals survive longer than the BCG parental strain immunized animals.

Primate safety and challenge study: More recently, the cynomolgus monkey has been used for evaluation of vaccines against M. tb. The evolutionary relationship between humans and non-human primates and the similar clinical and pathologic manifestations of tuberculosis in these species has made the non-human primate model attractive for experimental studies of TB disease and vaccine efficacy.

This model, characterized by the development of lung cavitation, appears to be applicable to human TB. The course of infection and disease is followed by X-ray and weight loss, as well as a variety of hematological tests, including erythrocyte sedimentation rate (ESR), peripheral blood mononuclear cell (PBMC) proliferation and cytokine production, cytotoxic T lymphocyte (CTL) activity, and antibody responses. Following infection, the cynomolgus monkey develops lung pathology with characteristic lesions, and, depending on the challenge doses, death from acute respiratory infection occurs within four-to six months after infection. Lower infection doses can lead to chronic infections without disease, much like in humans.

Study design The study directly compares varying doses of the BCG parental strain versus recombinant BCG administered either alone or followed by two subsequent boosters with the vaccine comprising sequences that are over expressed in the rBCG constructs. The latter may be delivered either as recombinant protein based in a suitable adjuvant formulation, DNA vaccine, or Ad35 (Adeno virus serum 35) constructs.

The first study evaluates the protective efficacy of the parental BCG vs rBCG constructs without a booster. This study comprises three groups (10 animals each) designed as follows: one group each comprising BCG, rBCG and saline. Two animals from each group are skin tested with the over expressed antigens in the rBCG constructs as well as with standard PPD and saline as controls. A positive and larger induration in the rBCG group compared with the BCG is indicative of in vivo vaccine take and the elicitation of an immune response. The remaining eight animals from each group are aerosol challenged with low dose M. tb Erdman strain and protection is measured by reduction of bacterial burden at 16 weeks post challenge or with survival as end point.

The follow up BCG prime protocol is essentially be the same as above except that the animals are first vaccinated with BCG, rBCG and saline followed by two boosters with the over-expressed antigens.

The immunogenicity and protection study in the non-human primate model investigates immunobiological and immunopathological aspects of tuberculosis in macaques for efficacy studies on rBCG constructs. The animals are juvenile to young adults raised in captivity with an average weight of 2 to 3 kg that have been thoroughly conditioned prior to the start of the experiment. Pre-inoculation studies consist of baseline blood tests that include routine hematological studies and erythrocyte sedimentation rates as well as lymphocyte proliferation assays. Skin testing is done with PPD to ensure lack of sensitivity to tuberculin and chest x-rays are obtained as part of the pre-infection profile. The immunization period lasts 21 weeks in total covering primary vaccination with BCG or rBCG at week=0, and antigen boosts at weeks 12 and 16. Antigen-specific immunity is assessed by measuring proliferation and interferon γ (IFN γ) secretion in lymphocyte stimulation tests. The frequency of IFNγ producing lymphocytes is determined by enzyme-linked immunosorbent assay (ELISPOT) or fluorescence-activated cell sorter (FACS). To this end, blood samples are drawn at weeks 0, 4, 8, 12, 16 and 20 weeks relative to primary vaccination.

Four to six weeks after the last immunization, animals are challenged by intratracheal installation of 3 ml (1,000 cfu) of the M. tuberculosis Erdman strain on the same day and with the same preparation. The course of the infection is assessed for weight loss, fever, elevated erythrocyte sedimentation rate (ESR), DTH to PPD, in vitro proliferative response of PBMC stimulated with PPD and the antigens over expressed in rBCG followed by measurements of the levels of IFN-g production. Chest x-rays are performed to detect abnormalities consistent with pulmonary TB, and finally, necropsy is carried out at 12-16 weeks post challenge.

Clinical evaluation of TB vectors and vaccines. Preclinical safety and toxicity studies as mandated by CBER guidelines and federal regulations are performed as preclinical toxicology and safety studies as described above. Following these studies human safety studies are performed. These studies are performed initially in healthy Quantiferon negative adults, and followed by age de-escalation into children and neonates.

Immunogenicity studies. Immunogenicity studies mice and primates utilizing but not limited to standard methods of evaluating cellular immunity such as INFγ ELISPOT, flow cytometry with short and long term antigen or peptide stimulation are employed. Similar methodologies are utilized for evaluation of human responses. Tetramer studies are employed for evaluation of CD4 and CD8 responses following vaccination of humans.

Optimization of prime-boost strategies. rBCG will work well as a stand alone vaccine against TB or other diseases for which it has been engineered to express relevant transgenes. A “transgene” as used herein is a DNA segment that is functionally linked to a mycobacterial promoter and expresses a protein of interest. rBCG as described here as a vaccine for TB or expressing transgenes to protect against other diseases also work extremely well to prime the immune system for booster immunization with recombinant proteins mixed with adjuvants or viral or bacterial vectored antigens. Both in animal preclinical studies and human studies, the BCG prime followed by recombinant protein/adjuvant or vector boosts are optimized in terms of regimens and doses. These prime boost strategies are the most potent means for inducing immunity in humans because of the potency of the BCG prime especially as embodied in this invention followed by focusing and enhancing the booster response of the immune system by recombinant protein or vector.

Clinical Evaluation of BCG Vectors

Oral administration of rBCG vaccines Oral vaccination of the target animal with the rBCG of the present invention may also be achieved using methods previously described (Miller et al., can Med Assoc J. 121(1): 45-54; 1979). The amount of the rBCG of the present invention administered orally will vary depending on the species of the subject, as well as the disease or condition that is being treated. Generally, the dosage employed is about 10³ to 10¹¹ viable organisms, preferably about 10⁵ to 10⁹ viable organisms.

The rBCG of this invention are generally administered along with a pharmaceutically acceptable carrier or diluent. The particular pharmaceutically acceptable carrier or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al, J. Clin. Invest, 79: 888-902; 1987); Black et al., J. Infect. Dis., 155: 1260-1265; 1987), or bicarbonate buffer (pH 7.0) containing ascorbic acid, lactose, and optionally aspartame (Levine et al, Lancet, II: 467-470; 1988). Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10% (w/v).

1. A Mycobacterium that is genetically engineered to remove its native super-oxide dismutase (Sod) enzyme and to contain and express a functional Sod enzyme from a heterologous bacterial genus, wherein said functional Sod enzyme from said heterologous bacterial genus is not secreted by said Mycobacterium.

2. The Mycobacterium of claim 1, wherein said functional Sod enzyme from a heterologous bacterial genus is isolated from a bacterial species selected from the group consisting of Salmonella enteriditis, Escherichia coli, Shigella flexneri, Listeria monocytogenes EGD-e, or a Corynebacterium species.

3. The Mycobacterium of claim 1, wherein said functional Sod enzyme from a heterologous bacterial genus is SodA from Listeria monocytogenes EGD-e.

4. The Mycobacterium of claim 1, wherein said Mycobacterium is an attenuated Mycobacterium.

5. The Mycobacterium of claim 4, wherein said attenuated Mycobacterium is BCG.

6. The Mycobacterium of claim 1, wherein said Mycobacterium further contains and expresses a transgene.

7. A method of decreasing the immunosuppressive properties of a Mycobacterium, comprising the step of
genetically engineering said Mycobacterium to remove its native super-oxide dismutase (SOD) enzyme and to contain and express a cytosol-bound Sod enzyme from a heterologous bacterial genus.

8. The method of claim 7, wherein said cytosol-bound Sod enzyme from a heterologous bacterial genus is isolated from a bacterial species selected from the group consisting of Salmonella enteriditis, Escherichia coli, Shigella flexneri, Listeria monocytogenes EGD-e and a Corynebacterium species.

9. The method of claim 8, wherein said cytosol-bound Sod enzyme from a heterologous bacterial genus is SodA from Listeria monocytogenes EGD-e.

10. The method of claim 7, wherein said Mycobacterium is an attenuated Mycobacterium.

11. The method of claim 10, wherein said attenuated Mycobacterium is BCG.

12. The method of claim 10, wherein said Mycobacterium is further genetically engineered to contain and express a functional transgene.

13. A vaccine preparation, comprising
a Mycobacterium that is genetically engineered to remove its native super-oxide dismutase (Sod) enzyme and to contain and express a functional super-oxide dismutase (Sod) enzyme from a heterologous bacterial genus, wherein said functional Sod enzyme from said heterologous bacterial genus is not secreted by said Mycobacterium.

14. The vaccine preparation of claim 13, wherein said functional Sod enzyme from said heterologous bacterial genus is isolated from a bacterial species selected from the group consisting of Salmonella enteriditis, Escherichia coli, Shigella flexneri, Listeria monocytogenes EGD-e or a Corynebacterium species.

15. The vaccine preparation of claim 14, wherein said functional Sod enzyme from said heterologous bacterial genus is SodA from Listeria monocytogenes EGD-e.

16. The vaccine preparation of claim 14, wherein said Mycobacterium is an attenuated Mycobacterium.

17. The vaccine preparation of claim 16, wherein said attenuated Mycobacterium is BCG.

18. The vaccine preparation of claim 16, wherein said Mycobacterium is further genetically engineered to contain and express a functional transgene.

19. The vaccine preparation of claim 16, wherein said Mycobacterium is genetically engineered to escape the endosomal compartment and enter the cytoplasm.

20. The vaccine preparation of claim 16, wherein said Mycobacterium is further genetic engineered to induce apoptosis.

21. The vaccine preparation of claim 16, wherein said Mycobacterium is further genetic engineered to express cytokines.

22. A method of treating cancer in a patient in need thereof, said method comprising the step of administering to said patient a vaccine preparation, comprising
a Mycobacterium that is genetically engineered to remove its native super-oxide dismutase (Sod) enzyme and to contain and express a functional super-oxide dismutase (Sod) enzyme from a heterologous bacterial genus, wherein said functional Sod enzyme from said heterologous bacterial genus is not secreted by said Mycobacterium.

23. The method of claim 23, wherein said functional Sod enzyme from said heterologous bacterial genus is SodA from Listeria monocytogenes EGD-e.

24. The method of claim 23, wherein said Mycobacterium is an attenuated Mycobacterium.

25. The method of claim 25, wherein said attenuated Mycobacterium is BCG.

26. The method of claim 23, wherein said Mycobacterium is further genetically engineered to contain and express a functional transgene.

27. The method of claim 23, wherein said Mycobacterium is genetically engineered to escape the endosomal compartment and enter the cytoplasm.

28. The method of claim 23, wherein said Mycobacterium is further genetic engineered to induce apoptosis.

29. The method of claim 23, wherein said Mycobacterium is further genetic engineered to express cytokines.
